(19) United States
(12) Patent Application Publication
(10) Pub. No.: US 2019/0290671 A1
(43) Pub. Date: Sep. 26, 2019

(54) HYPOLIPIDEMIC EFFECTS OF COMPOSITIONS COMPRISING BETA-GLUCOGALLIN

(71) Applicants: Muhammed Majeed, Edison, NJ (US); Kalyanam Nagabhushanam, East Windsor, NJ (US); Lakshmi Mundkur, Bangalore (IN); Shaheen Majeed, Springville, UT (US); Anurag Pande, East Windsor, NJ (US)

(72) Inventors: Muhammed Majeed, Edison, NJ (US); Kalyanam Nagabhushanam, East Windsor, NJ (US); Lakshmi Mundkur, Bangalore (IN); Shaheen Majeed, Springville, UT (US); Anurag Pande, East Windsor, NJ (US)

(21) Appl. No.: 16/361,457

(22) Filed: Mar. 22, 2019

Related U.S. Application Data
(60) Provisional application No. 62/647,905, filed on Mar. 26, 2018.

(51) Int. Cl. A61K 31/7034; A61P 3/06; A61K 31/05

(52) U.S. Cl. CPC A61K 31/7034; A61K 31/05; A61P 3/06

(57) ABSTRACT

Disclosed are the hypolipidemic effects of a composition comprising at least 10% w/w or above of 1-O-galloyl-β-D-glucose (β-glucogallin). The composition is effective in reducing the circulating levels of total cholesterol, triglycerides, LDL, cholesterol:HDL ratio and increasing HDL levels. The invention also discloses a method for reducing absorption and bio-accessibility of lipids in mammals using the abovementioned composition.

### CROSS-REFERENCE TO RELATED PATENT APPLICATIONS

This is a non-provisional filing claiming priority from U.S. Provisional Application No. 62/647,905 filed on 26 Mar. 2018

### BACKGROUND OF INVENTION

### Field of the Invention

The present invention relates to compositions comprising β-glucogallin for the therapeutic management of hyperlipidemia. More specifically, the present invention relates to the use of compositions comprising 10% β-glucogallin for effective control of lipid metabolism.

### Description of Prior Art

Hyperlipidemia is a condition caused due to aberrant lipid metabolism characterized by the presence of elevated circulating levels of cholesterol, low density lipoproteins (LDL), very low density lipoproteins (VLDL), triglycerides and lower levels of high density lipoproteins (HDL). It is considered as one of the risk factors for the development of diabetes, cardiovascular complications, atherosclerosis, hyperthyroidism or hypothyroidism, obesity, metabolic disorders, kidney diseases, neurodegenerative disorders, etc. Effective maintenance and management of circulating lipid levels is essential for maintaining a disease-free life.

Changes in lifestyle and diet help in managing hyperlipidemia effectively. Diet rich in fiber and omega-3-fatty acids, along with fruits and vegetables help in reducing cholesterol levels in blood. Apart from the lifestyle modifications different treatment methods are being employed for the management of hyperlipidemic conditions. Drugs like statins, cholesterol absorption inhibitors, such as ezetimibe, help in reducing the levels of circulating lipids. However, most of the drugs cause serious side effects like liver damage, muscle damage, type 2 diabetes and neurological dysfunction. Hence, a safe, effective and natural way for managing hyperlipidemia is warranted.

Natural molecules from plants sources have been reported to exhibit hypolipidemic effects (Tantawy and Temraz, Natural products for controlling hyperlipidemia: review, Arch Physiol Biochem. 2018; 19:1-8. doi: 10.1080/13813455.2018.1441315). But there still exists an unmet industrial need for a natural molecule that is effective in both preventing cholesterol absorption from the intestines and reducing circulating lipid levels. The present invention solves the abovementioned problem by disclosing a composition comprising β-glucogallin for reducing cholesterol absorption from intestines and lowering lipid levels in the blood.

It is the principle object of the invention to disclose a composition comprising β-glucogallin for reducing lipid absorption and accessibility.

It is yet another object of the invention to disclose a method for the therapeutic management of hyperlipidemia using a composition comprising β-glucogallin.

The invention solves the above mentioned objectives and provides further related advantages.

### SUMMARY OF THE INVENTION

The present invention relates to hypolipidemic effects of compositions comprising at least 10% w/w or above of 1-O-galloyl-β-D-glucose (β-glucogallin).

More specifically the invention discloses a method for reducing absorption and bio-accessibility of lipids in mammals using a composition comprising at least 10% w/w or above of 1-O-galloyl-β-D-glucose (β-glucogallin).

The invention also discloses a method for the therapeutic management of hyperlipidemia in mammals, said method comprising step of administering effective dose of a composition comprising at least 10% w/w or above of 1-O-galloyl-β-D-glucose (β-glucogallin), to mammals to bring about a reduction in the circulating levels of total cholesterol, triglycerides, LDL, cholesterol:HDL ratio and increasing HDL levels.

Other features and advantages of the present invention will become apparent from the following more detailed description, taken in conjunction with the accompanying images, which illustrate, by way of example, the principle of the invention.

### BRIEF DESCRIPTION OF THE DRAWINGS

FIG. 1 is a graphical representation showing the effect of composition comprising β-glucogallin on the lipolysis of fatty acids, monoglycerides, diglycerides and triglycerides.

FIG. 2 is a graphical representation showing the effect of composition comprising β-glucogallin on the bioaccessibility of cholesterol and saturated fatty acids.

FIG. 3 is a graphical representation showing the serum total cholesterol levels in rats administered with different concentrations of the composition comprising 1-glucogallin.

FIG. 4 is a graphical representation showing the serum triglyceride levels in rats administered with different concentrations of the composition comprising β-glucogallin.

FIG. 5 is a graphical representation showing the serum LDL levels in rats administered with different concentrations of the composition comprising β-glucogallin.

FIG. 6 is a graphical representation showing the serum HDL levels in rats administered with different concentrations of the composition comprising β-glucogallin

FIG. 7 is a graphical representation showing the serum cholesterol:HDL ratio in rats administered with different concentrations of the composition comprising 3-glucogallin

### DESCRIPTION OF PREFERRED EMBODIMENTS

In a most preferred embodiment, the invention relates to a method for reducing absorption and bio-accessibility of lipids in mammals, comprising step of administering effective dose of a composition comprising at least 10% w/w or above of 1-O-galloyl-β-D-glucose (β-glucogallin) to bring about a reduction in absorption and bio-accessibility of lipids. In a related aspect, the composition further comprises 10% w/w to 60% w/w total mucic acid gallates. In another related aspect, the mucic acid gallates include mucic acid 1,4-lactone 5-O-gallate, mucic acid 2-O-gallate, mucic acid 6-Methyl ester 2-O-gallate, mucic acid 1-Methyl ester 2-O-gallate and ellagic acid. In another related aspect, the lipid is selected from group comprising cholesterol, monoglycerides, diglycerides, triglycerides, lipoproteins, phospholipids, glycolipids, saturated fatty acids, unsaturated fatty acids, steroids and sphingolipids. In a preferred embodiment, the lipid is cholesterol and saturated fatty acids. In another related aspect, the composition does not impair fat digestion. In yet another related aspect, the effective dose is 250-500 mg. In another related aspect, the mammal is human.

In another preferred embodiment, the invention relates to a composition comprising at least 10% w/w or above of 1-O-galloyl-β-D-glucose (β-glucogallin) for use in decreasing the absorption and bio-accessibility of lipids. In a related aspect, the composition further comprises 10% w/w to 60% w/w total mucic acid gallates. In another related aspect, the mucic acid gallates include mucic acid 1,4-lactone 5-O-gallate, mucic acid 2-O-gallate, mucic acid 6-Methyl ester 2-O-gallate, mucic acid 1-Methyl ester 2-O-gallate and ellagic acid. In another related aspect, the lipid is selected from group comprising cholesterol, monoglycerides, diglycerides, triglycerides, lipoproteins, phospholipids, glycolipids, saturated fatty acids, unsaturated fatty acids, steroids and sphingolipids. In a preferred embodiment, the lipid is cholesterol and saturated fatty acids. In another related aspect, the composition does not impair fat digestion. In yet another related aspect, the effective dose is 250-500 mg. In another related aspect, the mammal is human.

In another related embodiment the invention relates to a method of therapeutic management of hyperlipidemia in mammals, comprising step of administering effective dose of compositions comprising at least 10% w/w or above of 1-O-galloyl-β-D-glucose (β-glucogallin) to bring about the effect of lowering levels of circulating cholesterol, LDL, triglycerides and cholesterol:HDL ratio and increasing HDL levels in the blood of said mammals. In a related aspect, the composition further comprises 10% w/w to 60% w/w mucic acid gallates. In another related aspect, the mucic acid gallates include mucic acid 1,4-lactone 5-O-gallate, mucic acid 2-O-gallate, mucic acid 6-Methyl ester 2-O-gallate, mucic acid 1-Methyl ester 2-O-gallate and ellagic acid. In yet another related aspect, the effective dose is 50-250 mg/kg body weight. In another related aspect, the composition is formulated with pharmaceutically/nutraceutically acceptable excipients, adjuvants, bases, diluents, carriers, conditioning agents, bioavailability enhancers, antioxidants and preservatives and administered orally in the form of tablets, capsules, syrups, gummies, powders, suspensions, emulsions, chewable, candies or eatables. In another related aspect, the mammal is human.

In another related aspect the invention relates to a composition comprising at least 10% w/w or above of 1-O-galloyl-β-D-glucose (β-glucogallin) for use in the therapeutic management of hyperlipidemia in mammals. In another related aspect, the composition further comprises 10% w/w to 60% w/w mucic acid gallates. In another related aspect, the composition reduces the levels of circulating cholesterol, LDL, triglycerides and cholesterol:HDL ratio and increases HDL levels in the blood of mammals. In a related aspect, the mucic acid gallates include mucic acid 1,4-lactone 5-O-gallate, mucic acid 2-O-gallate, mucic acid 6-Methyl ester 2-O-gallate, mucic acid 1-Methyl ester 2-O-gallate and ellagic acid. In yet another related aspect, the effective dose is 50-250 mg/kg body weight. In another related aspect, the composition is formulated with pharmaceutically/nutraceutically acceptable excipients, adjuvants, bases, diluents, carriers, conditioning agents, bioavailability enhancers, antioxidants and preservatives and administered orally in the form of tablets, capsules, syrups, gummies, powders, suspensions, emulsions, chewable, candies or eatables. In another related aspect, the mammal is human.

The following illustrative examples further explain the technical features and advantages of the present invention.

### EXAMPLES

### Example 1: Reduction in Lipid Absorption and Accessibility

Materials:

For the lipid digestion experiments Pancreatin from porcine pancreas, 4×USP specification, obtained from Sigma-Aldrich (Cat N P1750) was used. It contains pancreatic lipase and colipase at a molar ratio of 1:1 and a range of other enzymes, such as amylase, trypsin, ribonuclease and protease.

As a source of bile salts porcine bile extract was obtained from Sigma-Aldrich (Cat. no. B-8631), which contains 50 wt % bile acids, 6 wt % phosphatidylcholine and less than 0.06 wt % Ca2+. Gas chromatographic analysis showed that it contains also 1.24±0.18 wt % cholesterol and 6.7 wt % FA. According to the producer (personal communication), the composition of the bile salts in this extract is 13 wt % hyodeoxycholic acid, 18 wt % deoxycholic acid, 5 wt % cholic acid, 39 wt % glycodeoxycholic acid, and 24 wt % taurodeoxycholic acid. The percentages of these bile acids and the corresponding molecular masses were used to calculate an average molecular mass of 442 g·mol−1—the latter was used to define the average molar concentration of bile salts in our experiments.

Pepsin from porcine gastric mucosa (Fluka, Cat. no. 77160) was used in the “stomach” stage of the in vitro model.

All aqueous solutions were prepared using deionized water from the water-purification system Elix 3 (Millipore, USA). For preparation of electrolyte solutions, NaCl (product of Merck), KCl (Merck), CaCl₂) (Fluka) and NaHCO₃(Teokom), all of purity higher than 99%, were used.

Methods

Emulsion Preparation

Oil-in-water emulsion was used as a source of TG in the in vitro digestion experiments. The emulsion was from cocoa butter (primarily composed of saturated fatty acids) in the following way: first, the cocoa butter was melted at T=50° C., then 30 mL of it were added to 20 mL emulsifier solution, which was also thermostated at 50° C. Then, emulsification was performed with a rotor-stator homogenizer Ultra Turrax T25 (Janke & Kunkel GmbH & Co, IKA-Labortechnik), operating at 13 500 rpm for 5 min at T=50° C. The emulsifier solution contained 1 wt % surfactant Tween 80 (product of Sigma), 10 mM NaCl and 0.1 g L−1 NaN3 (as a preservative).

The drop size distribution in the emulsion was determined by video-enhanced optical microscopy. The diameters of the recorded oil drops were measured using custom-made image analysis software. For each sample, the diameters of at least 1000 drops were measured. The accuracy of these optical measurements was found to be ±0.3 μm. The mean drop size in the studied emulsions was characterized by the so-called volume-surface diameter, d32. The emulsion had d32=13±2 μm.

In Vitro Digestion Model

Briefly, the model consists of two stages, which stimulate the digestion in the stomach and in the small intestine. In the “stomach” stage, the pH is acidic (pH=1.3) and the protease pepsin is present. In the following “intestinal” stage, sodium bicarbonate was introduced to increase the pH to around 6.2 and then the bile extract and pancreatin (containing pancreatic lipase, proteases and other digestive enzymes) were added. The pH in the “intestinal” stage of the experiments increased gradually from 6.2 to 7.5 for 4 h, mimicking the pH-profile observed in vivo. The pepsin, bile salts and pancreatin solutions were prepared directly at 37° C., just before their use in the actual lipolysis experiments.

After a total reaction time of 4.5 h, the drug Orlistat (Xenical®, Roche) was added to inhibit completely the pancreatic lipase. Afterwards, the oil soluble components in the sample were extracted with chloroform or the sample was filtered to obtain a clear aqueous phase for further analysis of the lipids solubilized in the dietary mixed micelles (DMM).

Filtration

To analyse the lipid bioaccessbility at the end of the in vitro digestion experiment, the DMM from the much bigger oil droplets and solid precipitates was separated by filtration. The reaction mixture was first filtered through filter paper with a pore size of 2-3 μm and 84 g m-2 weight (BOECO, Germany). The filtration was carried out in a glass funnel and the filtrate was collected in a glass flask. Afterwards the obtained permeate was further filtered through a 200 nm nylon syringe filter (Minisart NY25, Sartorius, Germany). All procedures were performed at 37° C. The obtained permeate was clear and was then subjected to chloroform extraction.

Lipid Extraction by Chloroform

After stopping the lipolysis reaction with Orlistat granules, the reaction mixture was allowed to cool down to room temperature and its pH was decreased to pH=2 by adding HCl (to decrease the solubility of the fatty acids in the aqueous phase). Next, 6 mL chloroform was added and the sample was sonicated for 15 min. After every 5 min of sonication, the sample was vigorously agitated by shaking with hands. The obtained complex dispersion was centrifuged for 30 min at 3620 g (4500 rpm) which led to separation of clear aqueous and chloroform phases, indicating that the lipophilic substances were transferred into the non-polar phase. The obtained chloroform phase was further analyzed by Gas chromatography (GC) and the recovery of the cholesterol, fatty acids (FA), mono- and diglycerides (MG and DG), and tri-glycerides (TG) was found to be ≥90%. The same procedure was applied for analysis of the aqueous phase separated by filtration.

Gas Chromatography Analysis

The GC analyses were performed on a TRACE GC apparatus (ThermoQuest, Italy) equipped with autosampler AS 2000. We used a capillary column Thermo Fisher Scientific, USA, with the following specification: 5% phenyl methylpolysiloxane, 10 m length, I.D. 0.53 mm, 0.15 μm film thickness, high temperature (up to 400° C.). Cold on-column injection was used, at a secondary cooling time of 0.3 min.

The injection volume was 1 μL. The oven was programmed as follows: start at 120° C., hold 2 min, ramp 1 to 325° C. at 10° C./min, ramp 2 to 345° C. at 5° C./min, hold 5 min. The flame ionization detector (FID) temperature was set to 350° C. The carrier gas was helium, set at a constant pressure flow mode (60 kPa). The detector gases were hydrogen and air, with nitrogen as make-up gas. The secondary cooling gas was nitrogen with a purity of 99.99%. All other gases were of 99.999% purity. Before injection, the samples were derivatized by mixing with N,O-Bis (trimethylsilyl) trifluoroacetamide (BSTFA) for 1 h at 60° C.

The concentration of FA, MG, TG and cholesterol was calculated from the internal standard hexadecanol (cetanol), using correction factors of 1.16 and 1.90 for the FA and the TG, respectively. The correction factors were obtained from calibration curves with standard substances.

Experimental Results

The effect of composition containing β-glucogallin on the extent of lipid digestion was studied by in vitro digestion model and the results are presented in FIG. 1. 500 mg β-glucogallin had no significant effect on the concentration of any of the lipid digestion products (e.g. fatty acids, monoglycerides), compared to the control sample (in absence of β-glucogallin). Around 9 mM fatty acids, 3 mM monoglycerides amd <1 mM diglycerides are produced during the digestion of cocoa butter by the pancreatic enzymes, regardless of the presence of β-glucogallin. Less than 0.2 mM triglycerides remain undigested for both the control and β-glucogallin samples, which demonstrates the efficient in vitro digestion of cocoa butter at these conditions (degree of triglyceride lipolysis >95%).

To investigate if β-glucogallin affects lipid bioaccessibility, the samples were filtered in order to analyse only the lipid components solubilized in small dietary mixed micelles (DMM) that can easily penetrate the intestinal mucous layer and be absorbed in the blood stream.

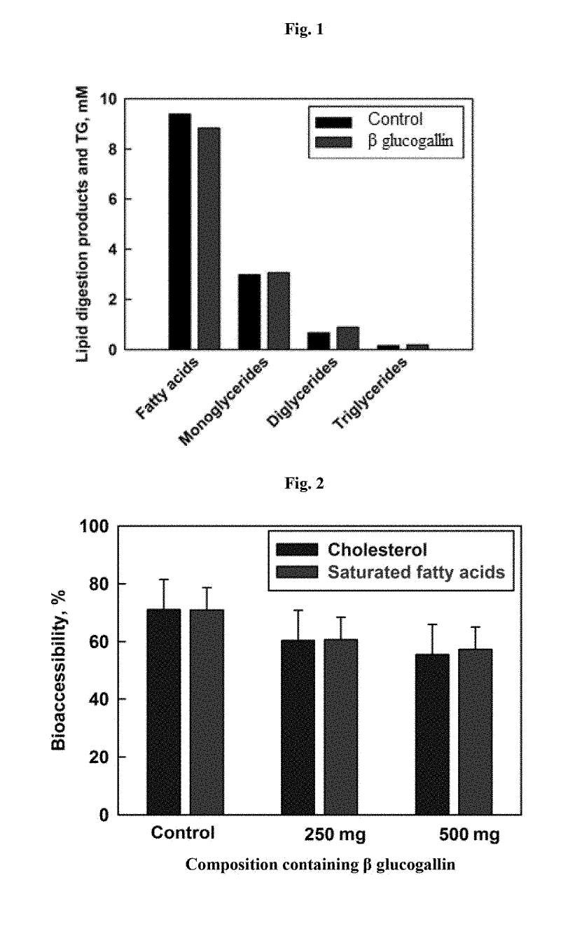

The obtained results for two concentration of a composition containing β-glucogallin, corresponding to 500 and 250 mg β-glucogallin are presented in FIG. 2. It was observed that β-glucogallin concentrations decrease the bioaccessibility of saturated fatty acids and cholesterol from ca. 70 to 60%. The latter results indicate that the ingestion of β-glucogallin could decrease the intestinal absorption of cholesterol and saturated fatty acids and in this way decrease the serum cholesterol in vivo.

Conclusion

The effect of composition comprising β-glucogallin on the extent of fat digestion and bioaccessibility of health-relevant compounds (cholesterol and saturated fatty acids) was studied. It was found that the β-glucogallin does not impair fat digestion, which remains very high: degree of triglyceride lipolysis >95%. At the same time, two concentrations of β-glucogallin, equivalent to 500 and 250 mg single intake, reduced the bioaccessibility of cholesterol and saturated fatty acids by 10%. Based on the above results we can conclude that β-glucogallin ingestion could potentially decrease serum cholesterol concentration and thus provide a health benefit, while being safe (does not alter normal digestion).

### Example 2: Hypolipidemic Effects of β-Glucogallin

Methodology

Test system:

| | |
|---|---|
| Animal Species: | Albino Rat |
| Strain: | Wistar |
| Sex: | Both Male |
| | and Female |
| Source: : | Biogen Laboratories |
| | Bangalore |
| No. of animals/group: | 6 animals/Group |
| | (3 Male and 3 Female). |
| Body weight | 100-120 g |
| range at receipt: | |
| Age at treatment: | 6-8 weeks |
| Identification: | Head, Body and Tail of |
| | both male and female |
| | mice were marked and |
| | kept separately. |

Performance of the Test
Feed: The animals were fed with Normal diet (9 kcal/day) and High fat diet (50 kcal/day) throughout the acclimatization and experimental period. The normal diet and High Fat diet were prepared as follows.

TABLE 1
Diet

| Ingredients | Normal Diet | | | High Fat Diet | | |
|---|---|---|---|---|---|---|
| Casein (g) | 20 | 80 | kcal | 20 | 80 | kcal |
| Sucrose (g) | 10 | 40 | kcal | 10 | 40 | kcal |
| Corn starch (g) | 20 | 80 | kcal | 25 | 100 | kcal |
| Lard (g) | 2 | 18 | kcal | 25 | 210 | kcal |
| Beef Tallow (g) | 2 | 18 | kcal | 20 | 180 | kcal |
| Mineral mix (g) | 23 | — | | 5 | — | |
| Vitamins (g) | 23 | — | | 5 | — | |
| Total | 100 g | 236 | kcal | 100 g | 610 | kcal |
| Total Energy Intake/Rat | | 23.6 | kcal | | 61 | kcal |
| (If Rat take 10 g/day) | | | | | | |

| | | |
|---|---|---|
| Total Fat Intake/ | 141.6 kcal | 366 kcal |
| 6 Rats/Group | | |

Water: Water was provided along with High Fat Diet to the animals throughout the acclimatization and experimental period. Water from water filter cum purifier was provided in animal feeding bottle with stainless steel sipper tubes.

Acclimatization:

The animals used for the present study were acclimatized for a period five days prior to initiate the experiment in laboratory condition and observed for clinical signs daily.

Study Design

TABLE 2
Test Substance-Composition containing β-glucogallin

| Groups | Treatment |
|---|---|
| ND | Normal Diet for 12 weeks |
| HFD | High Fat Diet for 12 weeks |
| HFD + BG 50 | High Fat Diet and treated with |
| | β-glucogallin composition |
| | (50 mg/kgbw) for 12 weeks |
| HFD + BG 100 | High Fat Diet and treated |
| | with β-glucogallin composition |
| | (100 mg/kgbw) for 12 weeks |
| HFD + BG 200 | High Fat Diet and treated with |
| | β-glucogallin composition |
| | (200 mg/kgbw) for 12 weeks |
| HFD + STATIN | High Fat Diet and treated with |
| | Atrovastin (3 mg/kgbw) for |
| | 12 weeks |

Body weight of the animals was recorded in 0^{th}, 15^{th}, 25^{th}, 40^{th}, 60^{th} and 84^{th} day of experimental period. At 0^{th} day, 1 ml Tail blood was collected from each animal of all the groups and serum was separated. At the end of the experimental period (84th day), the animals were sacrificed by cervical dislocation. Blood was collected and serum was separated by centrifugation and used for the analysis of biochemical parameters.

Route of Administration

The test item was administered through oral route by gavage.

Clinical Signs and Mortality

All the animals were observed once in a day for clinical signs and Mortality from 1^{st} day to 84^{th} day after dosing test item and standard drug during the study period. No mortality was observed among the animals during the experimental period.

Experimental Methods
Measurement of Body weight
Estimation of Cholesterol (CHOD/POD Method)
Estimation of Triglycerides (GPO/POD Method)
Estimation of HDL Cholesterol (HDL Cholesterol Direct Reagent Kit, Beacon)
Determination of LDL Cholesterol (LDL Cholesterol Direct Reagent Kit, Beacon)

Results

Body Weight

Significant increase in body weight was observed in with high fat diet feed. Dose dependent reduction in body weight was observed in the experimental animals (Group III to Group V) administered with HFD and β-glucogallin composition (Table 3)

TABLE 3
Effect of β-glucogallin on Body Weight of Experimental Animals (n = [6]).

| | Mean Body Weight (g) | | | | | |
|---|---|---|---|---|---|---|
| Groups | 0^{th} Day | 15^{th} Day | 25^{th} Day | 40^{th} Day | 60^{th} Day | 84^{th} Day |
| ND | 110.00 ± 8.94 | 121.66 ± 4.08 | 126.66 ± 4.08 | 147.50 ± 9.35 | 154.16 ± 10.20 | 161.66 ± 10.32 |
| HFD | 111.66 ± 8.16 | 137.50 ± 5.24 | 177.50 ± 8.21 | 220.83 ± 12.41 | 250.00 ± 13.03 | 278.33 ± 10.80 |
| HFD + BG 50 | 112.50 ± 9.35 | 130.00 ± 4.47 | 175.83 ± 13.19 | 207.50 ± 8.80 | 221.66 ± 8.75 | 232.50 ± 10.83 |
| HFD + BG 100 | 113.33 ± 8.75 | 128.33 ± 5.16 | 145.00 ± 15.81 | 182.50 ± 13.70 | 193.33 ± 11.69 | 203.33 ± 10.32 |
| HFD + BG 200 | 106.66 ± 6.05 | 128.33 ± 6.05 | 142.50 ± 5.24 | 159.16 ± 10.68 | 167.50 ± 10.83 | 176.66 ± 10.80 |
| HFD + STATIN | 108.33 ± 6.83 | 127.50 ± 6.90 | 139.16 ± 5.84 | 155.83 ± 7.35 | 165.83 ± 9.70 | 172.50 ± 9.35 |

Hypolipidemic Effects

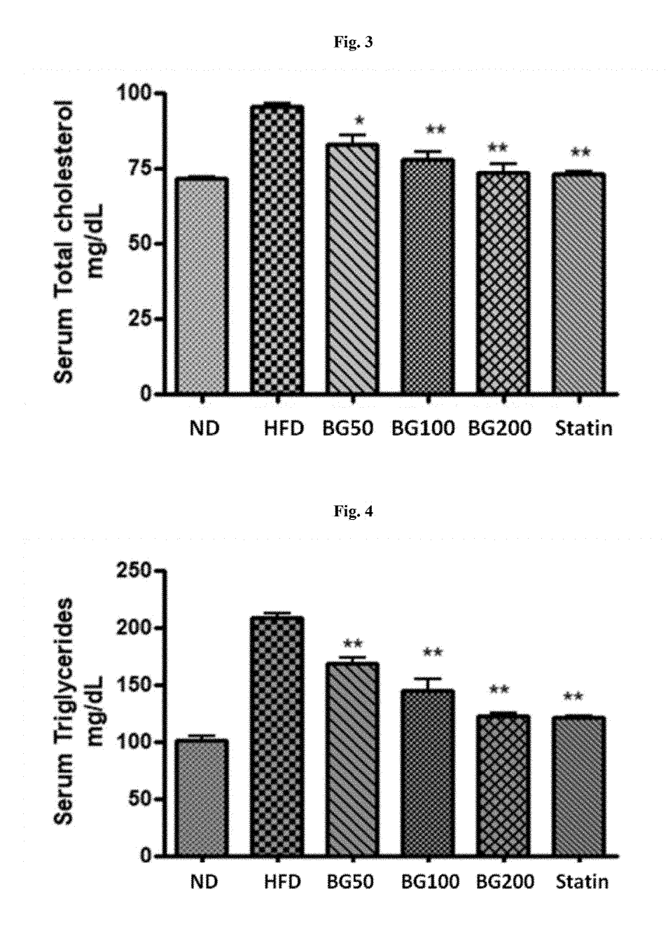

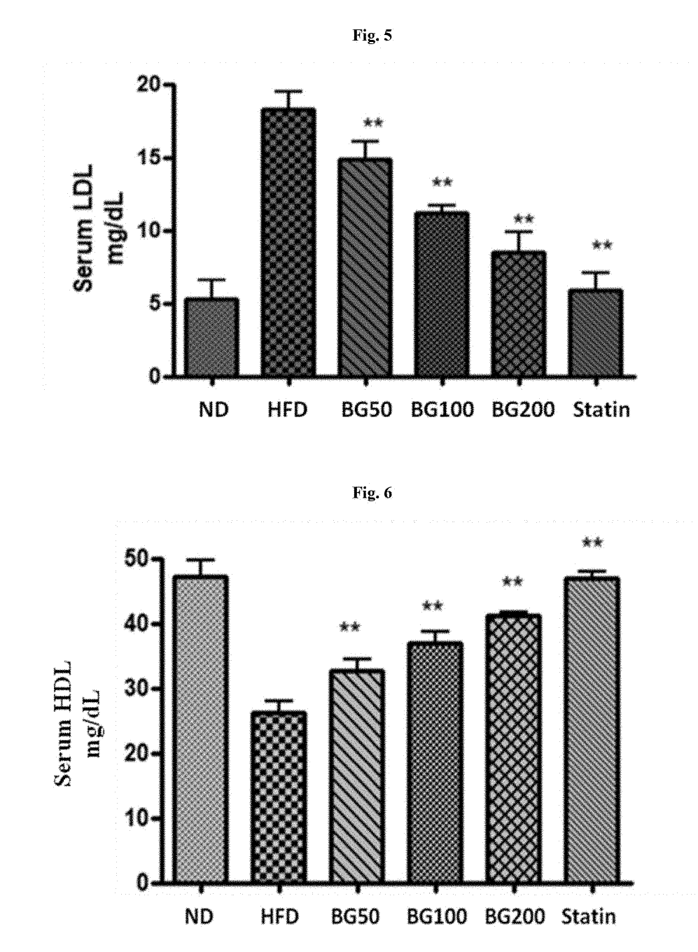

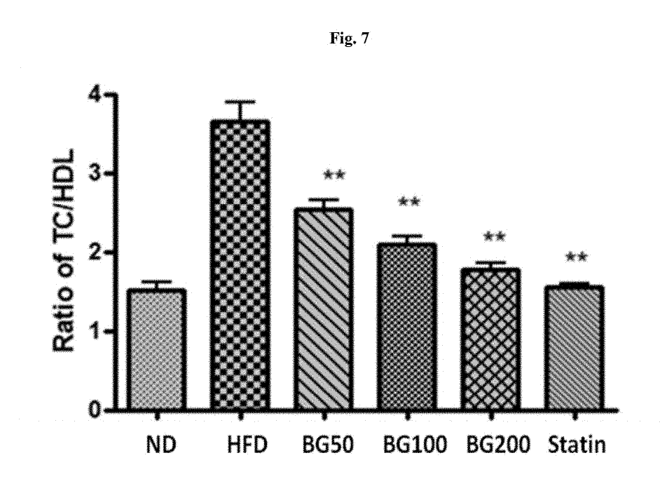

The lipid profile in the serum of the experimental animals were estimated using standard protocol. The total cholesterol levels in the HFD group were elevated compared to the control group. The composition comprising β-glucogallin, reduced the serum cholesterol levels in dose dependant manner (FIG. 3). Similarly, the serum triglycerides (FIG. 4) LDL levels were also elevated in the HFD group which was significantly lowered by i-glucogallin in a dose dependant manner. The composition containing β-glucogallin also increased the serum HDL levels (FIG. 5) normalizing the lipid levels in the blood. The cholesterol:HDL ratio was also reduced by the composition comprising β-glucogallin (FIG. 6).

From the data, it was concluded that, the intake of high fat diet for prolonged time affected lipid metabolism. β-glucogallin supplementation restored the aberrant lipid metabolism by normalizing the blood level of cholesterol, triglycerides, LDL and HDL.

### Example 3: Formulations Comprising β-Glucogallin

The composition comprising β-glucogallin is formulated with pharmaceutically/nutraceutically acceptable excipients, adjuvants, bases, diluents, carriers, conditioning agents, bioavailability enhancers, antioxidants and preservatives and administered orally in the form of tablets, capsules, syrups, gummies, powders, suspensions, emulsions, chewable, candies or eatables.

In a related aspect, one or more anti-oxidants and anti-inflammatory agents are selected from the group consisting of, but not limited to, vitamin A, D, E, K, C, B complex, rosmarinic acid, Alpha Lipoic Acid, oxyresveratrol, Ellagic Acid, Glycyrrhizinic Acid, Epigallocatechin Gallate, plant polyphenols, Glabridin, moringa oil, oleanolic acid, Oleuropein, Carnosic acid, urocanic acid, phytoene, lipoid acid, lipoamide, ferritin, desferal, billirubin, billiverdin, melanins, ubiquinone, ubiquinol, ascorbyl palmitate, Mg ascorbyl phosphate, ascorbyl acetate, tocopherols and derivatives such as vitamin E acetate, uric acid, α-glucosylrutin, calalase and the superoxide dismutase, glutathione, selenium compounds, butylated hydroxyanisole (BHA), butylated hydroxytoluene (BHT), sodium metabisulfite (SMB), propyl gallate (PG) and amino acid cysteine.

In another related aspect, one or more bioavailability enhancers are selected from the group, but not limited to, piperine, tetrahydropiperine, quercetin, Garlic extract, ginger extract, and naringin.

Tables 4-6 provide illustrative examples of composition containing β-glucogallin

TABLE 4
Rejuvenation blend
(Premix) containing
β-glucogallin
Active Ingredients
β-glucogallin and mucic acid
gallates (50-500 mg), Taurine
Excipients
Fructose, Citric Acid, Tartaric
Acid, Sucralose, Lemon
flavour and artificial
Peppermint Flavour

Directions for use: Dissolve the required premix in 200-300 ml water and mix well before use

TABLE 5
Digestive support blend
(Premix) containing
β-glucogallin
Active Ingredients
β-glucogallin and mucic
acid gallates (50-500 mg),
Bacillus coagulans
MTCC 5856,
Fenumannans, Triphala
Aquasol, 20% Gingerols,
Excipients
Maltodextrin, Citric Acid,
Malic Acid, Sucralose,
Lime, Spearmint and
Mangoginger flavours and
artificial Mint Flavour,
Cumin powder, Black Salt
powder, Asafoetida

Directions for use: Dissolve the required premix in 200-300 ml water and mix well before use

TABLE 6
Anti-oxidant/Cardiac
health blend (Premix)
containing β-glucogallin
Active Ingredients
β-glucogallin and mucic
acid gallates (50-500 mg),
Beetroot Extract, Citrin
Crystals (Anthocyanins),
Moringa Leaf Extract,
B Vitamins-Vitamin B6
and Vitamin B12,
Fruit Powder (Orange,
Banana, Pineapple, Apple,
Pomegranate, Jamun-
Cranberry, Grape)
Excipients
Maltodextrin, Xylitol,
Citric Acid, Malic Acid,
Potassium Chloride,
Sucralose, Calcium Silicate,
Strawberry flavour.

Directions for use: Dissolve the required premix in 200-300 ml water and mix well before use

Tables 7 and 8 provides illustrative examples of nutraceutical formulations containing β-glucogallin and mucic acid gallates for regulating lipid homeostasis

TABLE 7
β-glucogallin Tablet
Active Ingredients
β-glucogallin and
mucic acid gallates
(50-500 mg)
Excipients
Microcrystalline
cellulose,
Hypromellose,
Croscarmellose
Sodium, Colloidal
silicon dioxide,
Magnesium stearate

TABLE 8
β-glucogallin Capsule
Active Ingredients
β-glucogallin and
mucic acid gallates
(50-500 mg)
Excipients
Microcrystalline
cellulose,
Croscarmellose
Sodium,
Magnesium stearate

Table 9 provides illustrative example of a chewable gummy composition containing β-glucogallin and mucic acid gallates for regulating lipid homeostasis

TABLE 9
β-glucogallin
Gummy composition
Active Ingredients
β-glucogallin and mucic
acid gallates (50-500 mg),
Pectin, Glucose corn syrup
Excipients
Citiric acid, Lactic acid,
Lemon peel oil (flavor),
DL Tartaric acid,
refinated sugar

The above formulations are merely illustrative examples, any formulation containing the above active ingredient intended for the said purpose will be considered equivalent.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention.

We claim:

1. A method for reducing absorption and bio-accessibility of lipids in mammals, comprising step of administering effective dose of a composition comprising at least 10% w/w or above of 1-O-galloyl-β-D-glucose (β-glucogallin) to bring about a reduction in absorption and bio-accessibility of lipids.

2. The method for reducing absorption and bio-accessibility of lipids as in claim 1, wherein the composition further comprises 10% w/w to 60% w/w total mucic acid gallates

3. The composition as in claim 2, wherein the mucic acid gallates comprises mucic acid 1,4-lactone 5-O-gallate, mucic acid 2-O-gallate, mucic acid 6-Methyl ester 2-O-gallate, mucic acid 1-Methyl ester 2-O-gallate and ellagic acid.

4. The method as in claim 1, wherein the lipid is selected from group comprising cholesterol, monoglycerides, diglycerides, triglycerides, lipoproteins, phospholipids, glycolipids, saturated fatty acids, unsaturated fatty acids, steroids and sphingolipids.

5. The method as in claim 1, wherein the lipid is preferably cholesterol and saturated fatty acids.

6. The method as in claim 1, wherein the composition does not impair fat digestion.

7. The method as in claim 1, wherein the effective dose is 250-500 mg.

8. The method as in claim 1, wherein the mammal is human.

9. A method for therapeutic management of hyperlipidemia in mammals, comprising step of administering effective dose of compositions comprising at least 10% w/w or above of 1-O-galloyl-β-D-glucose (β-glucogallin) to bring about the effect of lowering levels of circulating cholesterol, LDL, triglycerides and cholesterol:HDL ratio and increasing HDL levels in the blood of said mammals.

10. The method for therapeutic management of hyperlipidemia as in claim 9, wherein the composition further comprises 10% w/w to 60% w/w total mucic acid gallates.

11. The method as in claim 10, wherein the mucic acid gallates comprises mucic acid 1,4-lactone 5-O-gallate, mucic acid 2-O-gallate, mucic acid 6-Methyl ester 2-O-gallate, mucic acid 1-Methyl ester 2-O-gallate and ellagic acid.

12. The method as in claim 9, wherein the effective dose is 50-250 mg/kg body weight.

13. The method as in claim 9, wherein the composition is formulated with pharmaceutically/nutraceutically acceptable excipients, adjuvants, bases, diluents, carriers, conditioning agents, bioavailability enhancers, antioxidants and preservatives and administered orally in the form of tablets, capsules, syrups, gummies, powders, suspensions, emulsions, chewable, candies or eatables.

14. The method as in claim 9, wherein the mammal is human.
